(19) United States
(12) Patent Application Publication
(10) Pub. No.: US 2002/0172988 A1
(43) Pub. Date: Nov. 21, 2002

(54) Nucleotide sequences

(76) Inventors: Jan Horling, Lidingo (SE); Magnus Jansson, Johanneshov (SE); Eva Danielsson, Hagersten (SE); Per Johansson, Kista (SE); Staffan Lake, Lidingo (SE); Joakim Nilsson, Danderyd (SE)

Correspondence Address: JANIS K. FRASER, PH.D., Fish & Richardson P.C., 225 Franklin Street, Boston, MA, 02110-2804, US

(21) Appl. No.: 10/145,949

(22) Filed: May 15, 2002

Related U.S. Application Data
(60) Provisional application No. 60/290,677, filed on May 15, 2001.

(30) Foreign Application Priority Data
May 15, 2001 (SE) 0101700-3

(51) Int. Cl. G01N 33/574; C07H 21/04; C12N 9/64; C12P 21/02; C12N 5/06

(52) U.S. Cl. 435/7.23; 435/69.1; 435/226; 435/320.1; 435/325; 536/23.2

(57) ABSTRACT

The present invention relates to human metalloprotease M8 polypeptides and to nucleic acid molecules coding for such polypeptides. The invention further relates to vectors and cells comprising the nucleic acid molecules, as well as to methods for screening for test compounds which affect the insulin-signaling pathway.

### CROSS REFERENCE TO RELATED APPLICATIONS

This application claims priority from Swedish Patent Application No. 0101700-3, filed May 15, 2001, and U.S. Provisional Patent Application Serial No. 60/290,677, filed May 15, 2001. These applications are incorporated herein by reference in their entirety.

### TECHNICAL FIELD

The present invention relates to metalloprotease M8 polypeptides and to nucleic acid molecules coding for such polypeptides. The invention further relates to vectors and cells comprising the nucleic acid molecules, as well as to methods for screening for test compounds that affect the insulin signaling pathway or glucose uptake.

### BACKGROUND ART

Insulin is the primary hormone involved in glucose homeostasis, and impairment of insulin action has a critical role in the pathogenesis of diabetes mellitus. In addition, insulin plays a key role in regulating a wide range of cellular processes. For reviews, see Shepherd, PR et al., (1998) Biochem. J. 333, 471-490; and Alessi, D. R. & Downes, C. P. (1998). Biochim. Biophys. Acta 1436, 151-164.

In order to find drug targets new research strategies have been developed. For instance, it has become clear that most genes in model organisms have counterparts in man and that many pathways, both metabolic and signaling, are conserved up to man. Well established genetics with attached methods have made it possible to analyze, in a systematic way, pathways in which known human gene homologues participate, genes which in man cause diseases such as diabetes.

In many diseases, responsible genes act by disturbing the normal homeostatic balance. This can be by over- or under-expression. Most, if not all, biological processes are under both positive and negative control and by that affected by several biochemical pathways and thus we can assume that control genes exist for all disease genes.

In many cases a drug interacts with a protein and either suppress or enhance its action. This can be mimicked in a genetic system and in an analysis thereof. Preferably, such an analysis can be done in organisms with a short generation time to allow a systematic finding of most or all these genes. One example of suitable model organism is the fruit fly, D. melanogaster, which have also the important feature of being an organism with a completely sequenced genome.

Therefore, the genetic analysis to identify genes, which when mutated lead to suppression or enhancement of the insulin signaling phenotype, gives a powerful tool to identify new and unknown drug targets.

The genetic screen initiate from a unique starting point established by modifying the insulin receptor in the insulin-signaling pathway. To perform genetic screens to identify candidate gene targets one can: (1) Generate and characterize misexpression and loss of function phenotypes in Drosophila and C. elegans suitable for genetic screens of the targeted pathways; (2) Perform genetic screens and epistasis analysis to identify genes that modify these phenotypes, and thus act in the pathway; and (3) Clone modifier genes and their human and mouse orthologues.

The goal of the insulin receptor signaling approach is to identify novel therapeutic entry points for the treatment of insulin resistance. The modulation of activity of a candidate therapeutic target should result in apparent enhancement of insulin receptor signaling through branches of the pathway associated with desirable metabolic outputs. Large-scale modifier screens are performed in Drosophila and yielded a large number of second-site (extragenic) mutations that uncovered previously unknown components of the insulin receptor pathway. The inventors have analyzed mutations that suppress or enhance the phenotypes caused by altered insulin signaling.

Modifiers that up-regulate the pathway (for example, suppressors of phenotypes caused by impaired signaling) will represent mutations in genes encoding proteins that normally down-regulate insulin receptor signaling. Vertebrate homologues of these genes are considered to be good targets for small molecule drugs that would modulate insulin responsiveness.

Suppressor mutations mimic the effect of inhibitory drugs on pathways that would normally act to attenuate the insulin response. Similarly, modifier mutations that down-regulate the pathway will represent genes whose wild-type function is to increase insulin signaling. Effects of these genes are tested in vertebrate tissue culture models after cloning of the vertebrate homologues. The vertebrate homologues would be candidate targets for pharmaceutical intervention. Novel genes identified in these screens are also candidates for disease associated genes or genes prognostic of clinical response to therapy.

P element mediated mutagenesis is a widely used technology in Drosophila genetics. The P element is a well-characterized transposable element, which can introduce heritable loss of function mutations into a wide array of genes. Coupled with genomic annotation of the P element insertion site, P element libraries provide a valuable reverse genetics tool. Genetic screens using libraries of P insertion mutants in known genes enable a rapid scanning of the genome to identify potential modifier genes.

Advanced P element vectors can also be used to test the effects of tissue specific over-expression of the nearby gene. In some instances, depending on insertion site, tissue specific antisense has also been reported. The inventors have currently performed a screen to identify modifiers of the InR eye phenotype using a library of over-expression P insertions. This approach rapidly identified a set of potential modifier genes, which can be further validated in invertebrate genetic assays or in vertebrate cell assays.

The protein IX-14 (D. melanogaster) has by bioinformatics been shown to be a metalloprotease of the M8 family and to be involved in insulin signaling, since over-expression results in insulin resistance (Exelixis). M8 proteases provisionally belong to the Clan MB, which also contains the families M6, M7, M10, M11 and M12. In families M10, M11 and M12 the third zinc ligand is His, whereas in families M6 and M7 it is Asp. Family M8 is included in clan MB primarily because of the mechanism of inhibition in the enzyme precursor. The M8 metalloprotease family has until now only been well characterized in protozoan parasites. Moreover, the M8 family contains leishmanolysin, the major cell surface protein in the promastigote stage of Leishmania and other flagellated, parasitic protozoa where it is essential for infection. In spite of the numerous investigations devoted to the characterization of leishmanolysin, the role of the enzyme in vivo remain unknown and is the subject of intense speculation. It has been demonstrated that the infectivity of the parasites can be inhibited or reduced by antibodies directed to Leishmanolysin.

Conclusively, in order to treat and cure diabetes and other insulin related diseases, a variety of drugs and drug targets are used. In the treatment of diabetes type II, one strategy is to inactivate a target, which in turn inactivates or attenuates the insulin-signaling pathway, thereby resulting in increased insulin sensitivity. In this sense, the M8 metalloprotease, being involved in insulin signaling, is an attractive target for finding drugs for insulin related diseases. However, a M8 molecule suitable as a drug target in the human body has until now not been found.

### SUMMARY OF THE INVENTION

Surprisingly, the inventors of the present invention have now discovered a human homologue to the metalloprotease M8 described above.

In a first aspect, the invention relates to a nucleic acid sequence encoding human metalloprotease M8. In a second aspect, the invention relates to the amino acid sequence of the human metalloprotease M8. Furthermore, the invention relates to recombinant vectors, cultured host cells, nucleic acid probes, antisense oligonucleotides, isolated antibodies and non-human transgenic animals related to the human metalloprotease M8 or its gene. Still further, the invention concerns methods for screening for compounds affecting the function of the human metalloprotease M8, as well as methods for disrupting the metalloprotease M8 gene in a non-human embryonic stem cell. The compounds affecting the function of the human metalloprotease M8 have surprisingly been found to affect the insulin signaling pathway, thereby being highly interesting potential drug targets.

The invention also covers variants of human metalloprotease M8 polypeptides derived from cDNA splice variants originating from the gene encoding said molecule. The human metalloprotease M8 polypeptides and all its variants are hereafter referred to as human metalloprotease M8.

The human M8 is not previously described in mammals as the metalloprotease M8. Moreover, the full-length sequence or correct sequence is not yet disclosed, and no function or pathway has been described. As the first M8 metalloprotease described in mammals, this invention not only open the gates to a new field of research, but also offers a potential opportunity to limit the effects of insulin resistance, and may also serve as a counter-screen protein for drugs targeting Leishmania.

### BRIEF DESCRIPTION OF THE DRAWINGS

FIG. 1 is a graph depicting the response of glucose oxidation on insulin stimulation of L6 cells transfected with a nucleic acid encoding M8.

FIG. 2A is a graph depicting the response of palmitate oxidation on insulin stimulation of 3T3-L1 cells transfected with a nucleic acid encoding M8.

FIG. 2B is a graph depicting the percent inhibition of palmitate oxidation in cells transfected with a nucleic acid encoding M8.

FIG. 3 is a graph depicting the glucose response of L6 cells transfected with different combinations of RNAi duplex and overexpressing vector.

### DETAILED DESCRIPTION OF THE INVENTION

The D. melanogaster protein IX-14 sequence was used to search human genomic and EST databases, using the BLAST algorithm (Altschul et al. (1997) Nucleic Acids Res. 25:3389-3402). A partial human metalloprotease M8 protein sequence was predicted from human genomic DNA sequence using GeneWise (Genome Res April 2000; 10(4):547-548). The predicted human M8 sequence is partially supported by known EST sequences. The human M8 protein sequence is 45% identical to the D. melanogaster protein IX-14 sequence and around 30% identical to protozoan M8 metalloproteases. The M8 proteases have a well conserved catalytic domain while the N- and C-terminal parts are less conserved.

In one aspect, the invention features an isolated nucleic acid molecule containing a nucleotide sequence that is at least 60% identical to the nucleotide sequence shown in SEQ ID NO:1, SEQ ID NO:3, SEQ ID NO:5, or SEQ ID NO:7. More preferably, the nucleotide sequence is at least 65%, 70%, 75%, 80%, 85%, 90%, 91%, 92%, 93%, 94%, 95%, 96%, 97%, 98%, 99%, or more, identical to the nucleotide sequence shown in SEQ ID NO:1, SEQ ID NO:3, SEQ ID NO:5, or SEQ ID NO:7. In the case of a nucleotide sequence that is longer than or equivalent in length to the reference sequence, e.g., SEQ ID NO:1, SEQ ID NO:3, SEQ ID NO:5, or SEQ ID NO:7, the comparison is made with the full length of the reference sequence. Where the nucleotide sequence is shorter that the reference sequence, e.g., shorter than SEQ ID NO:1, SEQ ID NO:3, SEQ ID NO:5, or SEQ ID NO:7, the comparison is made to a segment of the reference sequence of the same length (excluding any loop required by the homology calculation). Preferably, the nucleotide sequence encodes a polypeptide having protease activity. In one example, the nucleotide sequence is identical to SEQ ID NO:1, SEQ ID NO:3, SEQ ID NO:5, or SEQ ID NO:7.

In one embodiment, the invention features an isolated nucleic acid comprising a nucleotide sequence that encodes a polypeptide comprising an amino acid sequence that is at least about 60% identical to a sequence shown as SEQ ID NO:2, SEQ ID NO:4, SEQ ID NO:6, SEQ ID NO:8, or a fragment thereof. Preferably, the amino acid sequence is at least 65%, 70%, 75%, 80%, 85%, 90%, 95%, 98%, 99% or more identical to SEQ ID NO:2, SEQ ID NO:4, SEQ ID NO:6, or SEQ ID NO:8 and has a protease activity described herein. For example, the amino acid sequence can be identical to the sequence of SEQ ID NO:2, SEQ ID NO:4, SEQ ID NO:6, or SEQ ID NO:8. In one example, the polypeptide has a weight of about 77.3 kDa.

A sequential grouping of three nucleotides, a “codon,” codes for one amino acid. Since there are 64 possible codons, but only 20 natural amino acids, most amino acids are coded for by more than one codon. This natural “degeneracy”, or “redundancy”, of the genetic code is well known in the art. It will thus be appreciated that the nucleotide sequences of SEQ ID NO: 1, SEQ ID NO:3, SEQ ID NO:5, and SEQ ID NO:7 are only examples within a large but definite group of DNA sequences that encode the polypeptides described herein.

Also included in the invention is an isolated nucleic acid molecule comprising a nucleotide sequence that hybridizes under stringent hybridization conditions to the nucleotide sequence of SEQ ID NO:1, SEQ ID NO:3, SEQ ID NO:5, or SEQ ID NO:7, the complete complement of SEQ ID NO:1, SEQ ID NO:3, SEQ ID NO:5, or SEQ ID NO:7, or a segment thereof as described herein. The term “stringent hybridization conditions” is known in the art from standard protocols (e.g., Current Protocols in Molecular Biology, editors F. Ausubel et al., John Wiley and Sons, Inc. 1994, or Sambrook, J., Fritsch, E. F. and Maniatis, T., Molecular Cloning: A laboratory manual, 2nd Ed., Cold Spring Harbor Laboratory Press, Cold Spring Harbor, N.Y. 1989) and could be understood as e.g., hybridization to filter-bound DNA in 0.5 M NaHPO₄, 7% sodium dodecyl sulfate (SDS), 1 mM EDTA at +65° C., and washing in 0.1× SSC/0.1% SDS at +68° C. Preferably, the nucleotide sequence encodes a polypeptide having protease activity.

Also included in the invention is an isolated nucleic acid molecule comprising a nucleotide sequence that hybridizes under high stringency conditions to the nucleotide sequence of SEQ ID NO:1, SEQ ID NO:3, SEQ ID NO:5, or SEQ ID NO:7, the complete complement of SEQ ID NO:1, SEQ ID NO:3, SEQ ID NO:5, or SEQ ID NO:7, or a segment thereof as described herein. “High stringency conditions” refers to hybridization in 6× SSC at about 45° C., followed by one or more washes in 0.2× SSC, 0.1% SDS at 65° C. Preferably, the nucleotide sequence encodes a polypeptide having protease activity.

Also included in the invention is an isolated nucleic acid molecule comprising a nucleotide sequence that encodes a polypeptide comprising a functional domain of the polypeptide of SEQ ID NO:2, SEQ ID NO:4, SEQ ID NO:6, or SEQ ID NO:8 described herein, e.g., a catalytic domain of a metalloprotease M8 of SEQ ID NO:2, SEQ ID NO:4, SEQ ID NO:6, or SEQ ID NO:8. Preferably, the nucleotide sequence encodes a polypeptide having protease activity.

Also included in the invention is an isolated nucleic acid molecule comprising a nucleotide sequence that encodes a polypeptide comprising at least 20, 30, 40, 50, 75, 100, 150, 200, 250, 300, 350, 400, 450, 500, 550, 600, 650, or more contiguous amino acid residues of SEQ ID NO:2, SEQ ID NO:4, SEQ ID NO:6, or SEQ ID NO:8. In some embodiments, the polypeptide comprises an immunogenic fragment of at least 20 amino acids of SEQ ID NO:2, SEQ ID NO:4, SEQ ID NO:6, or SEQ ID NO:8. Preferably, the nucleotide sequence encodes a polypeptide having protease activity.

As used herein, an “isolated nucleic acid” is a nucleic acid, the structure of which is not identical to that of any naturally occurring nucleic acid or to that of any fragment of a naturally occurring genomic nucleic acid spanning more than three genes. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in random, uncharacterized mixtures of different DNA molecules, transfected cells, or cell clones, e.g., as these occur in a DNA library such as a cDNA or genomic DNA library.

As used herein, “% identity” of two amino acid sequences or of two nucleic acid sequences is determined using the algorithm of Karlin and Altschul (PNAS USA 87:2264-2268, 1990), modified as in Karlin and Altschul, PNAS USA 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12. BLAST protein searches are performed with the XBLAST program, score=50, wordlength=3. To obtain gapped alignment for comparison purposes GappedBLAST is utilized as described in Altschul et al (Nucleic Acids Res. 25:3389-3402, 1997). When utilizing BLAST and GappedBLAST programs the default parameters of the respective programs (e.g., XBLAST and NBLAST) are used to obtain nucleotide sequences homologous to a nucleic acid molecule of the invention.

In another aspect, the invention features a substantially pure polypeptide having a sequence shown as SEQ ID NO:2, SEQ ID NO:4, SEQ ID NO:6, or SEQ ID NO:8. The invention also includes a polypeptide, or fragment thereof, that differs from the corresponding sequence shown as SEQ ID NO:2, SEQ ID NO:4, SEQ ID NO:6, or SEQ ID NO: 8. The differences are, preferably, differences or changes at a non-essential residue or a conservative substitution. In one embodiment, the polypeptide includes an amino acid sequence at least about 60% identical to a sequence shown as SEQ ID NO:2, SEQ ID NO:4, SEQ ID NO:6, or SEQ ID NO:8, or a fragment thereof. Preferably, the amino acid sequence is at least 65%, 70%, 75%, 80%, 85%, 90%, 95%, 98%, 99% or more identical to SEQ ID NO:2, SEQ ID NO:4, SEQ ID NO:6, or SEQ ID NO:8 and has a protease activity described herein. For example, the amino acid sequence can be identical to SEQ ID NO:2, SEQ ID NO:4, SEQ ID NO:6, or SEQ ID NO:8.

Preferred polypeptide fragments of the invention are at least 10%, preferably at least 20%, 30%, 40%, 50%, 60%, 70%, or more, of the length of the sequence shown as SEQ ID NO:2, SEQ ID NO:4, SEQ ID NO:6, or SEQ ID NO:8 and have a protease activity described herein. Alternatively, the fragment can be merely an immunogenic fragment, e.g., a fragment that can be used to raise monoclonal and/or polyclonal antibodies that specifically bind to a polypeptide of the sequence of SEQ ID NO:2, SEQ ID NO:4, SEQ ID NO:6, or SEQ ID NO:8.

Included in the invention are variants, derivatives, and fragments of a metalloprotease M8 polypeptide. The skilled person will readily be able to determine whether such a variant, derivative, or fragment of a metalloprotease M8 polypeptide displays metalloprotease M8 activity by subjecting the variant, derivative, or fragment to a standard protease assay. Such assays are well known in the art, see e.g., Barrett, A. et al. Eds. Handbook of proteolytic enzymes, Academic Press 1998.

The invention encompasses polypeptides carrying modifications such as substitutions, small deletions, insertions or inversions, which polypeptides nevertheless have substantially the biological activities of a metalloprotease M8. Also included in the invention is a polypeptide encoded by a nucleic acid molecule described herein.

By a “functionally equivalent form” is meant a form of the protein, which possesses essentially the same activity, e.g., a protease activity, as a full length metalloprotease M8 polypeptide. A functionally equivalent form preferably comprises at least 300 amino acids, more preferably at least 500 amino acids, most preferably at least 600 amino acids.

Also included in the invention is a substantially pure polypeptide comprising a functional domain of the polypeptide of SEQ ID NO:2, SEQ ID NO:4, SEQ ID NO:6, or SEQ ID NO:8 described herein, e.g., a catalytic domain. Preferably, the polypeptide has protease activity.

Also included in the invention is a substantially pure polypeptide comprising at least 20, 30, 40, 50, 75, 100, 150, 200, 250, 300, 350, 400, 450, 500, 550, 600, 650, or more contiguous amino acid residues of SEQ ID NO:2, SEQ ID NO:4, SEQ ID NO:6, or SEQ ID NO:8. In some embodiments, the polypeptide comprises an immunogenic fragment of at least 20 amino acids of SEQ ID NO:2, SEQ ID NO:4, SEQ ID NO:6, or SEQ ID NO:8. Preferably, the polypeptide has protease activity.

The term “substantially pure” as used herein in reference to a given polypeptide means that the polypeptide is substantially free from other biological macromolecules. For example, the substantially pure polypeptide is at least 75%, 80, 85, 95, or 99% pure by dry weight. Purity can be measured by any appropriate standard method known in the art, for example, by column chromatography, polyacrylamide gel electrophoresis, or HPLC analysis.

The present invention also relates to vectors comprising the nucleic acid molecules of the invention, as well as host cells transformed with such vectors. Any of the nucleic acid molecules of the invention may be joined to a vector, which generally includes a selectable marker and an origin of replication for propagation in a host cell. Because the invention also provides polypeptides expressed from the nucleic acid molecules described above, vectors for the expression of a polypeptide are preferred. Such expression vectors include DNA encoding a polypeptide described herein, operably linked to suitable transcriptional or translational regulatory sequences, such as those derived from a mammalian, microbial, viral, or insect gene. Examples of regulatory sequences include transcriptional promoters, operators, or enhancers, mRNA ribosomal binding sites, and appropriate sequences, which control transcription and translation. Nucleotide sequences are operably linked when the regulatory sequence functionally relates to the DNA encoding a polypeptide. Thus, for example, a promoter nucleotide sequence is operably linked to a metalloprotease M8 DNA sequence if the promoter nucleotide sequence directs the transcription of the metalloprotease M8 sequence.

The vector may be any vector, which conveniently may be subjected to recombinant DNA procedures, and the choice of vector will often depend on the host cell into which it is to be introduced. Thus, the vector may be an autonomously replicating vector, e.g., a vector that exists as an extrachromosomal entity, the replication of which is independent of chromosomal replication (examples of such a vector are a plasmid, phage, cosmid, mini-chromosome or virus). Alternatively, the vector may be one which, when introduced in a host cell, is integrated in the host cell genome and replicated together with the chromosome(s) into which it has been integrated. Examples of suitable vectors are a bacterial expression vector and a yeast expression vector. The vector of the invention may carry any of the DNA molecules of the invention as defined above.

A suitable host cell can be a prokaryotic cell, a unicellular eukaryotic cell, or a cell derived from a multicellular organism. The host cell can thus, e.g., be a bacterial cell such as an E. coli cell, a cell from a yeast such as Saccharomyces cervisiae or Pichia pastoris, an insect cell, or a mammalian cell, such as HEK293, CHO or an equivalent. The methods employed to effect introduction of the vector into the host cell are standard methods well known to a person familiar with recombinant DNA methods.

Included in the invention is a process for production of a polypeptide described herein, which comprises culturing a host cell as described above under conditions whereby said polypeptide is produced, and optionally recovering said polypeptide, using standard biochemical procedures.

A further aspect of the invention is a nucleic acid probe comprising at least 15 nucleotides, which probe specifically hybridizes with at least a part of the nucleic acid molecule according to the invention, said part having a sequence shown in SEQ ID NO:1, SEQ ID NO:3, SEQ ID NO:5, or SEQ ID NO:7. The invention also provides an antisense oligonucleotide having a sequence capable of specifically hybridizing to at least a part of the nucleic acid molecule according to the invention, said part having a sequence shown in SEQ ID NO:1, SEQ ID NO:3, SEQ ID NO:5, or SEQ ID NO:7.

Fragments of the nucleic acid molecules described herein, as well as polynucleotides capable of hybridizing to such nucleic acid molecules may be used as a probe or as primers in a polymerase chain reaction (PCR). Such probes may be used, e.g., to detect the presence of nucleic acids coding for metalloprotease M8 in in vitro assays, as well as in Southern and Northern blots. Cell types expressing metalloprotease M8 may also be identified by the use of such probes. Such procedures are well known, and the skilled artisan will be able to choose a probe of a length suitable to the particular application. For PCR, 5′ and 3′ primers corresponding to the termini of a desired metalloprotease M8 nucleic acid molecule are employed to isolate and amplify that sequence using conventional techniques.

The polypeptides of the present invention may also be used to raise polyclonal and monoclonal antibodies, which are useful in diagnostic assays for detecting metalloprotease M8 polypeptide expression. Such antibodies may be prepared by conventional techniques. See, for example, Antibodies: A Laboratory Manual, Harlow and Land (eds.), Cold Spring Harbor Laboratory Press, Cold Spring Harbor, N.Y., (1988); Monoclonal Antibodies, Hybridomas: A New Dimension in Biological Analyses, Kennet et al. (eds.), Plenum Press, New York (1980).

Included in the invention is a method for screening for a compound that modulates (increases or decreases) an activity of a metalloprotease M8, the method comprising: contacting the a polypeptide described herein with a test compound; measuring a protease activity of the polypeptide in the presence of the test compound; comparing the protease activity of the polypeptide in the presence of the test compound with the protease activity of the polypeptide in the absence of the test compound, to thereby determine whether the test compound modulates an activity of a metalloprotease M8. The method can be used, for example, to screen for compounds that increase or decrease the effect or activity of metalloprotease M8, with the potential of alleviating pathogenic effects of defects in insulin signaling.

As a “test compound” is meant any suitable molecule being a potential drug target compound.

Furthermore, the invention provides a kit for carrying out the screening method above, comprising a polypeptide described herein, reagents for performing the method, and optionally instructions for use.

Included in the invention is a method of identifying an agent that binds to a metalloprotease M8, the method comprising: contacting a polypeptide comprising the amino acid sequence of SEQ ID NO:2, SEQ ID NO:4, SEQ ID NO:6, or SEQ ID NO:8 with a candidate agent; and determining that the candidate agent binds to the polypeptide, to thereby identify a candidate agent that binds to a metalloprotease M8.

The invention also provides a method for disrupting a metalloprotease M8 gene in a non-human embryonic stem cell, the method comprising: providing a nucleotide sequence (e.g., an oligonucleotide) capable of disrupting a metalloprotease M8 gene; and introducing the nucleotide sequence into a non-human embryonic stem-cell under conditions such that nucleotide sequence is homologously recombined into the metalloprotease M8 gene in the genome of the cell, to produce a cell containing at least one disrupted metalloprotease M8 allele. The invention further provides a non-human transgenic animal expressing reduced levels of a metalloprotease M8, wherein the metalloprotease M8 gene has been disrupted by the above method.

The invention also provides a non-human transgenic animal whose genome comprises an antisense nucleic acid molecule that hybridizes to an mRNA encoding a polypeptide described herein, thereby reducing translation of the polypeptide in the animal. A transgenic non-human mammal can e.g. be mammalian animal, such as a mouse, or an animal of the species Caenorhabditis elegans.

The invention also provides a method for screening for compounds that affect the insulin signaling pathway, the method comprising: providing a non-human transgenic animal described herein; providing a composition comprising a test compound in a form suitable for administration to the non-human animal; administering the test compound to the non-human animal; and determining the effect of the test compound on the insulin-signaling pathway in the animal. The method can be used to identify compounds that either increase or decrease the effect or activity of any molecule downstream of the insulin receptor, with the potential of alleviating the pathogenic effects of defects in insulin signaling.

Still further, the invention refers to the use of the substances found by the method of the invention for use in the treatment of diseases such as diabetes type 2, impaired glucose tolerance and obesity related insulin resistance. Impaired insulin resistance and associated glucose intolerance can occur in type 2 diabetes, type 1 diabetes, maturity-onset diabetes of the young (MODY), gestational diabetes, obesity and diseases related to the metabolic syndrome such dyslipedemia, atherosclerosis hypertension. Impaired insulin resistance and glucose intolerance can also occur in non-diabetic individuals and is considered as a predisposing factor for type 2 diabetes. Obesity is a condition in which there is an increase in body fat content resulting in excess body weight above accepted norms for age gender height and body build. An excess of mortality that occurs in obese individuals results from diseases that are predisposed by this condition. They include cancer, cardiovascular disease, digestion disease, respiratory disease and type 2 diabetes. In patients with chronic hyperglycaemia such as type 2 diabetes, glucose dependent protein cross-linking occurs at a rate in excess of the norm. Excessive non-enzymatic glycosylation of proteins contributes to diabetic complications and complications of aging in non-diabetic humans such as neuropathy, nephropathy, retinopathy, hypertension and atherosclerosis.

Reducing the insulin resistance and the abnormally high blood glucose level of diabetic subjects benefits the patient by reducing the discomfort of glucosuria and the excessively high mortality and morbidity associated with metabolic syndrome.

Leishmaniasis is a parasitic infection transmitted by the bite of an infected female sandfly whose hosts are animals, such as dogs or rodents, or human beings. The disease has four main forms, depending on the parasite species and the cellular immune system of the patient. Leishmaniasis currently threatens 350 million men, women and children in 88 countries around the world.

As declaration is obligatory in only 32 of the 88 countries affected by leishmaniasis, a substantial number of cases are never recorded. In fact, of the 1.5-2 million new cases estimated to occur annually, only 600,000 are officially declared. In addition, deadly epidemics of visceral leishmaniasis periodically flare up. For example, in the 1990s Sudan suffered a crisis with an excess mortality of 100,000 deaths among people at risk. An epidemic of cutaneous leishmaniasis is ongoing in Kabul, Afghanistan with an estimated 200,000 cases.

Leishmaniasis is spreading in several areas of the world as a result of epidemiological changes that sharply increase the overlapping of AIDS and visceral leishmaniasis. So far, 33 countries worldwide have reported co-infections. Leishmaniasis is one of the opportunistic infections that attack HIV-infected individuals, most of the co-infection involves the visceral form of leishmaniasis.

Treatment for co-infected patients is aimed at clinical and parasitological cures and prevention of relapses. Unfortunately, in such patient treatment failure, relapses due to drug resistance and drug toxicity are very common. In south-western Europe, follow-up studies using pentavalent antimonials, the same first-line drug used to treat classic leishmaniasis, show a positive response in 83% of cases. However, 52% of patients relapse within a period of one month to three years, with the number of relapses ranging from one to four. The main alternative drugs include pentamidine, amphotericin B and amphotericin B encapsulated in liposomes. This encapsulation reduces the occurrence of side effects, but relapses still occur and the drug remains extremely expensive.

Leishmanolysin (gp63) has been shown to be an important virulence factor; treatment of infective promastigotes with anti-gp63 antibodies inhibits intracellular parasite survival. Any substance inhibiting the activity of leishmanolysin or blocking its binding to host cell receptor is expected to reduce parasite infectivity. In order to treat patients with such a compound, a counter-screen using the human M8 should be considered.

In one aspect of the invention, such a counter-screen may be performed in a method for screening for compounds that modulate protease activity, the method comprising: contacting a test compound to a protease compound; determining the effect of the test compound on a protease activity of the protease compound; contacting the test compound to a polypeptide described herein; and determining the effect of the test compound on a protease activity of the polypeptide, to thereby screen for compounds that modulate protease activity. The protease compound can be any suitable protease compound other than the polypeptide (e.g., a metalloprotease M8 polypeptide). Preferably, the protease compound is leishmanolysin, or any other functionally equivalent compound.

A purpose of the counter-screen is to find compounds showing a positive effect (e.g., inhibition) on the protease compound, such as leishmanolysin, and negative effect (e.g., no inhibition) on the polypeptide, e.g., a metalloprotease M8 polypeptide. Such a compound can constitute a potential drug target for leishmaniasis, but not having a specific effect on the insulin signaling pathway.

Furthermore, the invention relates to the use of substances found by the methods of the invention for the manufacture of a medicament for treating the diseases or states in the group comprising type 2 diabetes, type 1 diabetes, MODY, gestational diabetes, obesity, dyslipedemia, atherosclerosis, hypertension, cancer, cardiovascular disease, digestive disease, respiratory disease, neuropathy, nephropathy, retinopathy, hypertension, atherosclerosis and leishmaniasis.

Further, the combination of the methods described in example 4A and 4B can form the basis for large scale screening of compound libraries as methods for developing specific inhibitors of metalloprotease M8.

A major outcome of the sequence and activity information of metalloprotease M8 is the development of an orally administered chemical entity that itself, or metabolites thereof, specifically inhibits the proteolytic activity of metalloprotease M8. Such an entity can be used to treat diabetic states in human.

The availability of active recombinant human metalloprotease M8 is used in the development of screening tools used for developing compounds for inhibition of activity. The homology of metalloprotease M8 with known metalloproteases from protozoans, e.g., Leishmania ssp. is important in developing screens for inhibition of cell surface metalloproteases known to be important for parasite lifecycle.

The amino acid coding region of the M8 cDNA is cloned in its entirety. The deduced sequence contains a translation initiation methionine codon as well as in-frame translation stop sequence.

The rationale behind metalloprotease M8 as a target in insulin signaling pathway and the potential treatment of diabetic states in man is based on inhibition of protease activity. Gene knockout or antisense expression in animal models with a diabetic phenotype gives potential for measuring the potentiation of the phenotype as well as reversal of phenotype when treated with metalloprotease M8 inhibitory compounds.

Unless otherwise defined, all technical and scientific terms used herein have the same meaning as commonly understood by one of ordinary skill in the art to which this invention belongs. Suitable methods and materials are described below, although methods and materials similar or equivalent to those described herein can also be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

Below, the invention is described in the appended examples, which are intended to illustrate the invention, without limiting the scope of protection.

### EXAMPLES

### Example 1

### Isolation of cDNA Clone

It has been shown by Exelixis in their screen of D. melanogaster expressing a dominant negative insulin receptor that over-expression of IX-14 results in a weak but consistent enhancement of the phenotype. This leads to the interpretation that sense expression of the IX-14 metalloprotease results in increased insulin resistance.

To isolate a cDNA clone encoding M8, the predicted DNA sequence (SEQ ID NO:1) based on proprietary data (INCYTE/CELERA) was used to design PCR primers for 5′-RACE cDNA amplification using the SMART RACE cDNA Amplification Kit, Clontech (cat #K1811-1). PCR primers are shown as SEQ ID NO:9-11.

As template for cloning, a 5′-RACE-Ready cDNA preparation was made on human pancreas mRNA, Clontech (cat. # 6539-1) according to the manual. 1 μg of mRNA was used for the reaction and Superscript II reverse transcriptase, Gibco BRL (cat #18064-22), was used for the reverse transcription. The 5′-RACE, 50 μl PCR-reaction, was made according to the manual using 5 μl universal primer mix (UPM), and 1 μl PCR primer. The PCR reactions were performed using Advantage2 Polymerase Mix, Clontech (cat # 8430-2). The PE GeneAmp System 9700 was used with the program: 94° C. 20 sec, 25 cycles of 94° C. 5 sec, 72° C. 3 minutes and finally 72° C. 7 minutes and cooling to 4° C.

20 μl were loaded on a 0.8% low gel agarose (BioRad) and a band of about 2 kbp was seen in the 5′ PCR reaction. The fragment was cut out from the gel and 2,5 μl of the fragment was cloned into 1 μl plasmid pCR2.1-TOPO using the TOPO TA Cloning Kit, Invitrogen (cat #K4500-01). 5 μl of the ligation mix was transformed into One Shot chemically competent TOP10 E. coli cells, Invitrogen (cat #C4040-03).

Plasmid DNA from four colonies (30 ml overnight culture), were obtained by using the QIAGEN Midiprep (cat #12143) and the subsequent sequencing was performed by the dideoxy chain-termination method using Applied Biosystems Model AB1377 DNA sequencing system.

### Example 2A

### Northern Blotting

A probe for northern blot analysis was obtained from the cloned M8 by a PCR reaction with primers shown as SEQ ID NOS:10 and 11 which gave a fragment of 340 bp in the catalytic domain of the gene. The PCR reactions was performed using Advantage cDNA polymerase mix from Clontech with the following PCR program: +94° C. for 30 seconds, +54° C. for 30 seconds, +72° C. for 1 minute for 30 cycles, with an initial step of +94° C. for 3 minutes and a last step of +72° C. for 7 minutes. A Perkin Elmer 9700 machine was used. The band of interest was excised from agarose gel and purified by using QIAquick Gel Extraction Kit (QIAGEN). The probe was radioactively labeled according to the rediprime kit (Amersham Pharmacia Biotech). Human Multiple Tissue Northern (MTNBlots (Clontech) was used for hybridization of the cDNA probe as described in the MTN Blots protocol.

### Example 2B

### Tissue Specific Transcript Profiling

A probe for transcript profiling analysis was obtained from the cloned M8 by a PCR reaction with primers shown as SEQ ID NOS:9 and 10 which gave a fragment of 340 bp in the catalytic domain of the gene. The PCR reactions was performed using Advantage cDNA polymerase mix from Clontech with the following PCR program: +94° C. for 30 seconds, +54° C. for 30 seconds, +72° C. for 1 minute for 30 cycles, with an initial step of +94° C. for 3 minutes and a last step of +72° C. for 7 minutes. A Perkin Elmer 9700 machine was used. The band of interest was excised from agarose gel and purified by using QIAquick Gel Extraction Kit (QIAGEN). The probe was radioactively labeled according to the rediprime kit (Amersham Pharmacia Biotech). Human Multiple Tissue Expression (MTE™) Array blots was used for hybridization of the cDNA probe as described in the MTN Blots protocol.

### Example 3

### Use of Antisense Oligonucleotides for Producing Knock-Out Animals

Knowledge of the cDNA sequence of metalloprotease M8 will enable its use as tool in sense (Youssoufan et al. (1993) Mol. Cell. Biol. 13, 98-104) or antisense (Eguchi et al. (1991) Ann. Rev. Biochem. 60, 631-652) technologies for the investigation of gene function. Oligonucleotides, from genomic or cDNAs, comprising either the sense or the antisense strand of cDNA sequence can be used in vivo or in vitro to inhibit expression. Such technology is well known in the art, and oligonucleotides or other fragments can be designed from various locations along the sequences. The gene of interest can be turned off in the short term by transfecting a cell or tissue with expression vectors which will flood the cells with sense or antisense sequences until all copies of the vector are disabled by endogenous nucleases.

### Example 4

### Protease Analysis

4A. Determination of Substrate Specificity

The proteolytic specificity of the described metalloprotease M8 is determined by measuring the cleavage of synthetic peptide substrates based on the sequence information derived from molecules known to be substrates to homologues of the same protease class. The resulting cleavage products are characterized by MALDI-TOF mass spectrometry analysis. The resulting cleaved peptide fragments gives the preferred cleavage specificity of metalloprotease M8.

4B. Determination of Specific Activity

Measurement of specific activity can be determined using a fluorescence release assay based on the specificity of the metalloprotease M8. Fluorescence labeled peptides is used to monitor the activity. Bound AMC is released upon protease cleavage and the resulting increase in fluorescence is determined. Fluorescence release based assays are well known in the art and can be combined with information from Example 4A for the specific activity determination.

The following Examples 5 and 6 were performed using over-expression of human M8.

### Example 5

Glucose oxidation in L6 cells transfected with M8 36 T-25 flasks containing differentiated L6 cells were transfected with M8 (15.6 μg DNA/flask, DNA: Lipofectamine 1:2.5) and incubated for 48 hours prior glucose oxidation rate determinations. The transfected cells were then serum starved for 5 hours. A medium (3.0 ml) supplemented with ¹⁴C-glucose (0.5 μCi/ml) was added to each flask, and a device containing filter paper was mounted in the cultivation flasks, the screw caps were tightened and the cells were incubated for 1 hour at 37° C.

Prior to termination of the CO₂ measurements, 0.3 ml of hyamine hydroxide solution was applied on filter paper and 0.4 ml of 4 M sulphuric acid was injected through a septum into the medium. The flasks were then incubated for additional 60 minutes at room temperature. Finally the filters were cut into small pieces and transferred into scintillation vials. Scintillation solution (10 ml) and methanol (200 μl) was added to each vial and the samples were counted for radioactivity.

Blanks were prepared by processing flasks containing medium (no cells) exactly as described above.

Transfection Procedure

Flasks with differentiated L6 cells were washed in serum-free/low glucose αMEM and then incubated with 15.6 μg DNA and 39 μl lipofectamine/flask (DNA/lipofectamine were prepared according to manufacturer's instructions) in serum-free/low glucose αMEM. After 5 hours, an equal volume (1.5 ml/flask) of αMEM supplemented with 20% fetal calf serum was added and the incubation was continued for additional 19 hours. The medium was subsequently replaced with low glucose αMEM supplemented with 10% fetal calf serum (2001-10-23), and the cells were assayed 24 hours later.

Glucose Oxidation

Cells were serum starved in 0.25% BSA, αMEM for 18 hours prior addition of a tracer.

The cells in an aspirate medium, were washed with glucose/serum-free DMEM, and then incubate for 60 minutes in glucose/serum-free DMEM supplemented with 200 nM insulin (when indicated) and tracer.

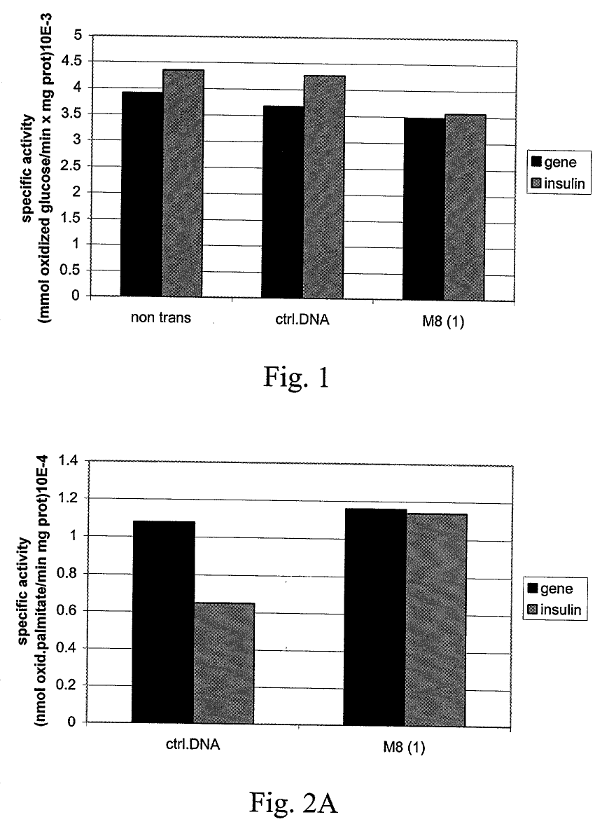

Procedure for incubation media; to 140 ml of glucose/serum-free DMEM, serum free, low glucose medium were added 280 μl of (U)-¹⁴C-glucose. This medium was divided in to 2 parts of 70 ml each. In the first part no insulin were added (see series “gene” in FIG. 1). In the second part (see series “insulin” in FIG. 1) 16.8 μl of 5 mg/ml insulin was added to a final concentration of 200 nM (1.2 μg/ml). Insulin additions are done at time 0. The results are shown in FIG. 1, where the two columns to the left, marked “non trans”, represent non transfected cells that contains no plasmid vector, the two columns in the middle, marked “ctrl.DNA”, represent cells transfected using same vector as in gene specific experiments however lacking the gene of interest, also referred to as “backbone” vector; and the two columns to the right, marked “M8(1)”, represents cells transfected with M8.

The conclusion from this example is that insulin stimulation increase the glucose oxidation in non-transfected cells and in cell control transfected with carrier DNA vector. Cell transfected with and over-expressing M8 has a decreased response to insulin stimulation. Thus M8 is involved in down-regulating insulin response.

### Example 6

Palmitate Oxidation in 3T3-L1 Cells

36 T-25 flasks containing differentiated 3T3 cells were transfected with constructs containing the gene of interest (31.3 μg DNA/flask, DNA: Lipofectamine 1:2.5) and incubated for 48 hours prior palmitate oxidation rate determinations.

Cells were serum starved for 5 hours. 3.0 ml medium supplemented with ¹⁴C-palmitate (0.5 μCi/ml) was added to each flask, and a device containing filter paper was mounted in the cultivation flasks, the screw caps were tightened and cells were incubated for 1 hour at 37° C.

Prior to termination of the CO₂ measurements, 0.3 ml of hyamine hydroxide solution was applied on filter paper and 0.4 ml of 4 M sulphuric acid was injected through a septum into the medium. The flasks were then incubated for additional 60 minutes at RT. Finally the filters were cut into small pieces and transferred into scintillation vials. Scintillation solution (10 ml) and methanol (200 μl) was added to each vial and the samples were counted for radioactivity.

Blanks were prepared by processing flasks containing medium (no cells) exactly as described above.

Transfection Procedure

Flasks with differentiated 3T3-L1 cells were washed in serum-free/low glucose DMEM and than incubated with 31.3 μg DNA and 78.1 μl lipofectamine/flask (DNA/lipofectamine were prepared according to manufacturer's instructions) in serum-free/low glucose DMEM. After 3 hours, en equal volume (1.5 ml/flask) of DMEM supplemented with 20% fetal calf serum was added and the incubation was continued for additional 19 hours. The medium was subsequently replaced with low glucose DMEM supplemented with 10% fetal calf serum (2001-09-18), and cells were assayed 24 hours later.

Palmitate Oxidation

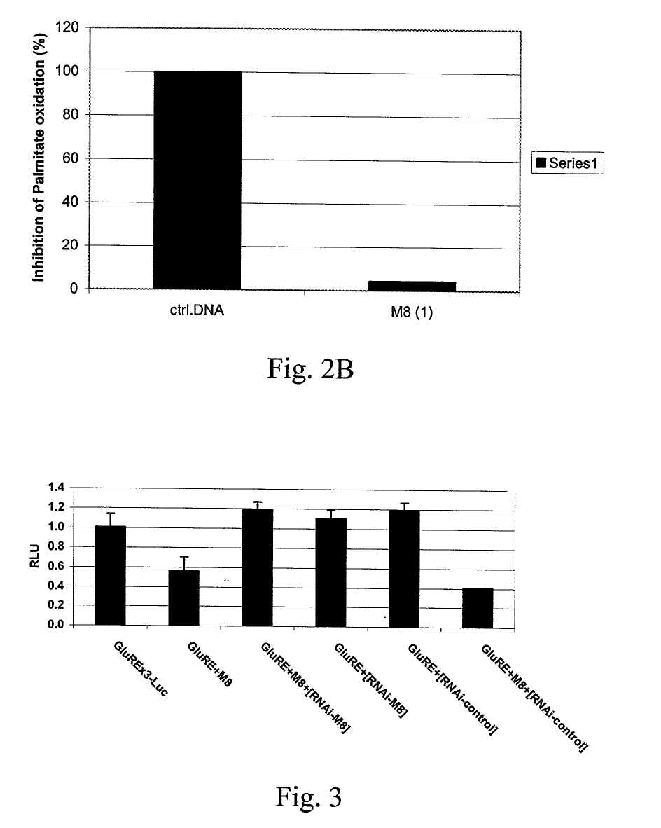

Cells were serum starved for 5 hours prior addition of the tracer. Procedure for incubation media: To 120 ml of DMEM, serum free, low glucose medium were added 500 μl of (U)-¹⁴C-palmitate. This medium was divided to 2 parts of 60 ml each. To the first part no insulin was added (see series “gene” in FIG. 2A). To the second part 60 μl of 0.1 mg/ml insulin was added (see series “insulin” in FIG. 2A). Insulin additions are done at time 0.

The response of palmitate on insulin stimulation of 3T3-L1 cells transfected with M8 is shown in FIG. 2A, where the two left hand columns, marked “ctrl.DNA”, represent cells transfected using same vector as in gene specific experiments however lacking the gene of interest; and the two right hand columns, marked “M8(1)”, represents cells transfected with M8.

The percent inhibition of palmitate oxidation is shown in FIG. 2B, were the control transfected response, ctrl.DNA, is set to 100%.

The conclusion from this experiment is that control cells stimulated with insulin shows a decreased palmitate oxidation due to higher glucose uptake and thus an increase in glucose oxidation. Cells over-expressing M8 shows a decreased response to insulin the palmitate oxidation is almost unaffected due to a down-regulated glucose uptake.

### Example 7

RNAi L6 gluRE

The following example was performed using suppression of M8 expression. Differentiated skeletal muscle cells (L6) were transiently transfected with a glucose response element coupled to a luciferase reporter gene (GluREx3-Luciferase), M8 and RNAi-M8, using LipofectAmine2000 (Invitrogen).

Per well in a 96-well plate a total of 0.4 μg DNA was used (0.2 μg reporter gene construct and 0.07 ug gene of interest or backbone plasmid and 0.13 μg RNAi) and 2.25 μl lipid reagent/ug DNA diluted in 50 μl Opti-MEM (Gibco). Methods for preparing the gene construct are described by Elbashir et al., (2001) Nature 411, 494-498; and Harborth et al.; (2001) J Cell Sci. 114(Pt 24), 4557-4565. Sequences used for RNAi mediated gene silencing were SEQ ID NO:14 and SEQ ID NO:15. These oligos are reverse complimentary and form a double stranded RNA/DNA hybrid.

The cells were incubated 4 hrs with the DNA/lipid reagent complex. After 48 hrs the cells were lysed using 15 μl/well of lysis buffer (TRIS-EDTA+0,25% Triton-x100) and the luciferase activity was measured by adding 50 μl of each ATP and luciferin (Luciferase activity kit, BioThema). The Relative Luminesence Units (RLU) for GluREx3-Luciferase was set to 1 and the other values were normalized to this value. The results are shown in FIG. 3, where the columns from left to right respectively represents:

cells transfected with GluREx3-Luc reporter gene vector;

cells transfected with GluREx3-Luc reporter gene vector and M8 over-expressing vector;

cells transfected with GluREx3-Luc reporter gene vector, M8 over-expressing vector and anti M8 RNAi duplex;

cells transfected with GluREx3-Luc reporter gene vector and anti M8 RNAi duplex;

cells transfected with GluREx3-Luc reporter gene vector, a control vector and anti M8 RNAi duplex, i.e. the same as for the third column from the left, but not containing the gene of interest; and

cells transfected with GluREx3-Luc reporter gene vector, M8 over-expressing vector and RNAi duplex against non related sequence.

The conclusion from Example 7 is that RNAi methodology down-regulates the expression of the targeted gene, here M8. The GluRE reporter gene assay is proportional to the amount of glucose taken up by the cell. Over-expression of M8, bar 2, decreases the response due to decreased glucose uptake. Down regulation of both over-expressed and endogenous M8, bar 3 and 4, restore the original response. Using RNAi targeted at an independent gene, bar 6, does not restore the activity. The restored glucose response is thus dependent on the specific decrease of M8.

### Example 8

### Identification of M8 Splice Variants

To isolate further cDNA transcripts encoding M8, the predicted DNA sequence based on proprietary data (INCYTE/CELERA) and the sequence derived from 5′-RACE cDNA amplification, primers were designed (SEQ ID NOS:12 and 13).

As template for the PCR 0.5 μl human Retina QUICK-Clone cDNA (1 ng/μl, Clontech Laboratories, Palo Alto, Calif., USA) was used. The following conditions were used for the PCR-reaction, 10 pmol of primer-mix JN18/JN20 and JN18/JN21 respectively, 1 μl dNTPs (10 mM), 1 μl Pfu Turbo DNA Polymerase (Stratagene, La Jolla, Calif., USA), 5 μl 10× Pfu DNA Polymerase reaction buffer (Clontech) in a total volume of 50 μl. Amplification was performed with a Perkin-Elmer 2400 thermocycler (Perkin-Elmer, Norwalk, Conn., USA). The PCR-program consisted of an initial denaturation at 94° C. for 1 minute, 35 cycles of 94° C. for 30 s, 63° C. for 30 seconds and 72° C. for 3 minutes followed by a final extension at 72° C. for 10 minutes. The Pfu Turbo DNA Polymerase was added during the initial 94° C. denaturation step. The PCR-products were cloned into the vector pCR-BluntII-TOPO (Invitrogen, Carlsbad, Calif.). The cloned PCR-fragments were sequenced in both directions according to a standard protocol for dye terminator cycle sequencing and analyzed on a DNA sequencer ABI 377 (Applied Biosystems, Foster City, Calif., USA).

From the molecular cloning of M8, four different splice variants (SEQ ID NOS:1, 3, 5 and 7) were detected. Each of the sequences encode a variant polypeptide chain (SEQ ID NOS:2, 4, 6 and 8) derived from differential splicing of the transcript encoded by the genomic sequence. Screening of the assembled human genomic sequence indicates that it contains only one possible gene encoding for these sequences. Consequently, the detected forms must be derived by differential splicing from the same pre-mRNA transcript.

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

What is claimed is:

1. An isolated nucleic acid molecule comprising a nucleotide sequence that is at least 85% identical to the sequence of SEQ ID NO:1, SEQ ID NO:3, SEQ ID NO:5, or SEQ ID NO:7.

2. The nucleic acid molecule of claim 1, wherein the nucleotide sequence encodes a polypeptide having protease activity.

3. The nucleic acid molecule of claim 1, wherein the nucleic acid molecule comprises the sequence of SEQ ID NO:1, SEQ ID NO:3, SEQ ID NO:5, or SEQ ID NO:7.

4. An isolated nucleic acid molecule comprising a nucleotide sequence that encodes a polypeptide comprising an amino acid sequence that is at least 85% identical to the sequence of SEQ ID NO:2, SEQ ID NO:4, SEQ ID NO:6, or SEQ ID NO:8.

5. The nucleic acid molecule of claim 4, wherein the polypeptide has protease activity.

6. An isolated nucleic acid comprising a nucleotide sequence that encodes a polypeptide comprising the amino acid sequence of SEQ ID NO:2, SEQ ID NO:4, SEQ ID NO:6, or SEQ ID NO:8.

7. An isolated nucleic acid molecule comprising a nucleotide sequence that hybridizes under stringent hybridization conditions to the sequence of SEQ ID NO:1, SEQ ID NO:3, SEQ ID NO:5, SEQ ID NO:7, or a complete complement thereof.

8. The nucleic acid molecule of claim 7, wherein the nucleotide sequence encodes a polypeptide having protease activity.

9. An isolated nucleic acid molecule comprising a nucleotide sequence that encodes a polypeptide comprising at least 100 contiguous amino acid residues of SEQ ID NO:2, SEQ ID NO:4, SEQ ID NO:6, or SEQ ID NO:8.

10. An isolated nucleic acid molecule comprising a nucleotide sequence that encodes a polypeptide comprising an immunogenic fragment of at least 20 amino acids of SEQ ID NO:2, SEQ ID NO:4, SEQ ID NO:6, or SEQ ID NO:8.

11. A substantially pure polypeptide comprising an amino acid sequence that is at least 85% identical to the sequence of SEQ ID NO:2, SEQ ID NO:4, SEQ ID NO:6, or SEQ ID NO:8.

12. The polypeptide of claim 11, wherein the polypeptide has protease activity.

13. A substantially pure polypeptide comprising the amino acid sequence of SEQ ID NO:2, SEQ ID NO:4, SEQ ID NO:6, or SEQ ID NO:8.

14. The polypeptide of claim 13, wherein the polypeptide consists of the amino acid sequence of SEQ ID NO:2, SEQ ID NO:4, SEQ ID NO:6, or SEQ ID NO:8.

15. A substantially pure polypeptide comprising at least 100 contiguous amino acid residues of SEQ ID NO:2, SEQ ID NO:4, SEQ ID NO:6, or SEQ ID NO:8.

16. A substantially pure polypeptide comprising an immunogenic fragment of at least 20 amino acids of SEQ ID NO:2, SEQ ID NO:4, SEQ ID NO:6, or SEQ ID NO:8.

17. A vector comprising the nucleic acid molecule of claim 1.

18. A replicable expression vector comprising the nucleic acid molecule of claim 1 operably linked to a regulatory element that directs expression of the nucleic acid molecule.

19. A cultured host cell comprising the vector of claim 17.

20. A method for producing a polypeptide, the method comprising culturing the host cell of claim 19 under conditions whereby the polypeptide is produced.

21. A nucleic acid probe comprising at least 15 nucleotides, wherein the probe specifically hybridizes to at least a part of the nucleic acid molecule of claim 3, said part having a sequence shown as in SEQ ID NO:1, SEQ ID NO:3, SEQ ID NO:5, or SEQ ID NO:7.

22. An antisense oligonucleotide having a sequence that specifically hybridizes to at least a part of the nucleic acid molecule of claim 3, said part having a sequence shown as in SEQ ID NO:1, SEQ ID NO:3, SEQ ID NO:5, or SEQ ID NO:7.

23. A pair of primers comprising a first primer and a second primer, wherein the first primer hybridizes to the sense strand of the nucleic acid molecule of claim 3, and wherein the second primer hybridizes to a strand complementary to the sense strand of the nucleic acid molecule.

24. An isolated antibody that specifically binds to the polypeptide of claim 14.

25. A method for screening for a compound that modulates an activity of a metalloprotease M8, the method comprising:
contacting the polypeptide of claim 11 with a test compound;
measuring a protease activity of the polypeptide in the presence of the test compound;
comparing the protease activity of the polypeptide in the presence of the test compound with the protease activity of the polypeptide in the absence of the test compound,
to thereby determine whether the test compound modulates an activity of a metalloprotease M8.

26. A kit for carrying out the method of claim 25, the kit comprising a substantially pure polypeptide comprising an amino acid sequence that is at least 85% identical to the sequence of SEQ ID NO:2, SEQ ID NO:4, SEQ ID NO:6, or SEQ ID NO:8, and instructions for use.

27. A method of identifying an agent that binds to a metalloprotease M8, the method comprising:
contacting a polypeptide comprising the amino acid sequence of SEQ ID NO:2, SEQ ID NO:4, SEQ ID NO:6, or SEQ ID NO:8 with a candidate agent; and
determining that the candidate agent binds to the polypeptide,
to thereby identify a candidate agent that binds to a metalloprotease M8.

28. A method for disrupting a metalloprotease M8 gene in a non-human embryonic stem cell, the method comprising:
providing a nucleotide sequence capable of disrupting a metalloprotease M8 gene; and
introducing the nucleotide sequence into a non-human embryonic stem-cell under conditions such that nucleotide sequence is homologously recombined into the metalloprotease M8 gene in the genome of the cell, to produce a cell containing at least one disrupted metalloprotease M8 allele.

29. A non-human transgenic animal expressing reduced levels of metalloprotease M8, wherein the metalloprotease M8 gene has been disrupted by the method of claim 28.

30. A non-human transgenic animal whose genome comprises an antisense nucleic acid molecule that hybridizes to an mRNA encoding the polypeptide of claim 4, thereby reducing translation of the polypeptide in the animal.

31. The non-human transgenic animal of claim 30, wherein the animal is a mouse.

32. The non-human transgenic animal of claim 30, wherein the animal is of the species Caenorhabditis elegans.

33. A method for screening for compounds that affect the insulin signaling pathway, the method comprising:
providing the non-human transgenic animal of claim 29;
providing a composition comprising a test compound in a form suitable for administration to the non-human animal;
administering the test compound to the non-human animal; and
determining the effect of the test compound on the insulin-signaling pathway in the animal.

34. A method for screening for compounds that modulate protease activity, the method comprising:
contacting a test compound to a protease compound;
determining the effect of the test compound on a protease activity of the protease compound;
contacting the test compound to the polypeptide of claim 11; and
determining the effect of the test compound on a protease activity of the polypeptide,
to thereby screen for compounds that modulate protease activity.

35. The method of claim 34, wherein the protease compound is leishmanolysin.
